(19) United States
(12) Patent Application Publication
(10) Pub. No.: US 2022/0084653 A1
(43) Pub. Date: Mar. 17, 2022

(54) METHOD FOR GENERATING IMAGE OF ORTHODONTIC TREATMENT OUTCOME USING ARTIFICIAL NEURAL NETWORK

(71) Applicant: HANGZHOU ZOHO INFORMATION TECHNOLOGY CO., LTD., Hangzhou City (CN)

(72) Inventor: LINGCHEN YANG, Hangzhou City (CN)

(21) Appl. No.: 17/531,708

(22) Filed: Nov. 19, 2021

Related U.S. Application Data
(63) Continuation-in-part of application No. PCT/CN2020/113789, filed on Sep. 7, 2020.

(30) Foreign Application Priority Data
Jan. 20, 2020 (CN) 202010064195.1

(51) Int. Cl. G16H 20/40; G06T 17/00; G06T 7/73; G06T 7/11; G06K 9/00; G06T 7/00; G16H 30/20; G06N 3/08; A61C 7/00

(52) U.S. Cl. CPC G16H 20/40; G06T 17/00; G06T 7/75; G06T 7/11; G06K 9/00281; G06T 7/0016; G06T 2210/41; G06N 3/08; A61C 7/002; G06T 2207/20084; G06T 2207/30036; G06T 2207/30201; G16H 30/20

(57) ABSTRACT

In one aspect of the present application, a method for generating image of orthodontic treatment outcome using artificial neural network is provided, the method comprises: obtaining a picture of a patient's face with teeth exposed before an orthodontic treatment; extracting a mouth mask and a first set of tooth contour features from the picture of the patient's face with teeth exposed before the orthodontic treatment using a trained feature extraction deep neural network; obtaining a first 3D digital model representing an initial tooth arrangement of the patient and a second 3D digital model representing a target tooth arrangement of the patient; obtaining a first pose of the first 3D digital model based on the first set of tooth contour features and the first 3D digital model; obtaining a second set of tooth contour features based on the second 3D digital model at the first pose; and generating an image of the patient's face with teeth exposed after the orthodontic treatment using a trained deep neural network for generating images, based on the picture of the patient's face with teeth exposed before the orthodontic treatment, the mask and the second set of teeth contour features.

### CROSS REFERENCE TO RELATED APPLICATIONS

The present application is a continuation-in-part application of International (PCT) Patent Application No. PCT/CN2020/113789, filed on Sep. 7, 2020, which claims priority to Chinese Patent Application No. 202010064195.1, filed on Jan. 20, 2020, the disclosure of which is incorporated by reference herein.

### FIELD OF THE APPLICATION

The present application generally relates to a method for generating image of orthodontic treatment outcome using artificial neural network.

### BACKGROUND

Nowadays, more and more people get to know that orthodontic treatment is not only good for health but also improves aesthetic appearance. For a patient who is unfamiliar with orthodontic treatment, if appearance of teeth and face after a treatment is shown to the patient before the treatment, this may help the patient to build confidence in the treatment, and meanwhile this may promote communications between the dentist and the patient.

Currently, there is no solution for generating image of orthodontic treatment outcome. A conventional technique using 3D model texture mapping usually cannot generate high quality and lifelike presentations. Therefore, it is necessary to provide a method for generating image of patient's appearance after orthodontic treatment.

### SUMMARY

In one aspect, the present application provides a method for generating image of orthodontic treatment outcome using artificial neural network, which comprises: obtaining a picture of a patient's face with teeth exposed before an orthodontic treatment; extracting a mouth mask and a first set of tooth contour features from the picture of the patient's face with teeth exposed before the orthodontic treatment using a trained feature extraction deep neural network; obtaining a first 3D digital model representing an initial tooth arrangement of the patient and a second 3D digital model representing a target tooth arrangement of the patient; obtaining a first pose of the first 3D digital model based on the first set of tooth contour features and the first 3D digital model; obtaining a second set of tooth contour features based on the second 3D digital model at the first pose; and generating an image of the patient's face with teeth exposed after the orthodontic treatment using a trained deep neural network for generating images, based on the picture of the patient's face with teeth exposed before the orthodontic treatment, the mask and the second set of tooth contour features.

In some embodiments, the deep neural network for generating images may be a CVAE-GAN network.

In some embodiments, a sampling method used by the CVAE-GAN network may be a differentiable sampling method.

In some embodiments, the deep neural network for generating images includes a decoder, where the decoder may be a StyleGAN generator.

In some embodiments, the feature extraction deep neural network may be a U-Net network.

In some embodiments, the first pose may be obtained using a nonlinear projection optimization method based on the first set of tooth contour features and the first 3D digital model, and the second set of tooth contour features may be obtained by projecting the second 3D digital model at the first pose.

In some embodiments, the method for generating image of orthodontic treatment outcome using artificial neural network may further comprise: segmenting a first image of mouth region from the picture of the patient's face with teeth exposed before the orthodontic treatment using a face key point matching algorithm, where the mouth mask and the first set of tooth contour features are extracted from the first image of mouth region.

In some embodiments, the picture of the patient's face with teeth exposed before the orthodontic treatment may be a picture of the patient's full face.

In some embodiments, the contour of the mask matches the contour of the inner side of the lips in the picture of the patient's face with teeth exposed before the orthodontic treatment.

In some embodiments, the first set of tooth contour features may comprise outlines of teeth visible in the picture of the patient's face with teeth exposed before the orthodontic treatment, and the second set of tooth contour features may comprise outlines of the second 3D digital model at the first pose.

In some embodiments, the tooth contour features may be a tooth edge feature map.

### BRIEF DESCRIPTION OF THE DRAWINGS

The above and other features of the present disclosure will be understood more sufficiently and clearly through the following description and appended claims with reference to figures. It should be understood that these figures only depict several embodiments of the content of the present disclosure, so they should not be construed as limiting the scope of the content of the present disclosure. The content of the present disclosure will be illustrated in a more definite and detailed manner by using the figures.

FIG. 1 schematically illustrates a flow chart of a method for generating an image of a patient's appearance after an orthodontic treatment using artificial neural network in one embodiment of the present application;

FIG. 2 schematically illustrates a first image of mouth region in one example of the present application;

FIG. 3 schematically illustrates a mask generated based on the first image of mouth region shown in FIG. 2 in one embodiment of the present application;

FIG. 4 schematically illustrates a first tooth edge feature map generated based on the first image of mouth region shown in FIG. 2 in one embodiment of the present application;

FIG. 5 schematically illustrates a block diagram of a feature extraction deep neural network in one embodiment of the present application;

FIG. 5A schematically illustrates the structure of a convolutional layer of the feature extraction deep neural network shown in FIG. 5 in one embodiment of the present application;

FIG. 5B schematically illustrates the structure of a deconvolutional layer of the feature extraction deep neural network shown in FIG. 5 in one embodiment of the present application;

FIG. 6 schematically illustrates a second tooth edge feature map in one embodiment of the present application;

FIG. 7 schematically illustrates a block diagram of a deep neural network for generating images in one embodiment of the present application; and

FIG. 8 schematically illustrates a second image of mouth region in one embodiment of the present application.

### DETAILED DESCRIPTION OF ILLUSTRATED EMBODIMENTS

In the following detailed description, reference is made to the accompanying drawings, which form a part thereof. In the figures, like symbols usually represent like parts, unless otherwise additionally specified in the context. Exemplary embodiments in the detailed description, figures and claims are only intended for illustration purpose and not meant to be limiting. Other embodiments may be utilized and other changes may be made, without departing from the spirit or scope of the present disclosure. It will be readily understood that aspects of the present disclosure generally described in the text herein and illustrated in the figures can be arranged, replaced, combined and designed in a wide variety of different configurations, all of which are explicitly contemplated and make part of the present disclosure.

After extensive research, the Inventors of the present application discovered that as the deep learning technology arises, generative adversarial networks are already able to generate images that can pass for real pictures in some fields. However, the orthodontic field still lacks a robust solution for generating images based on deep learning. After a lot of works on designing and tests, the Inventors of the present application have developed a method for generating an image of a patient's appearance after an orthodontic treatment using artificial neural network.

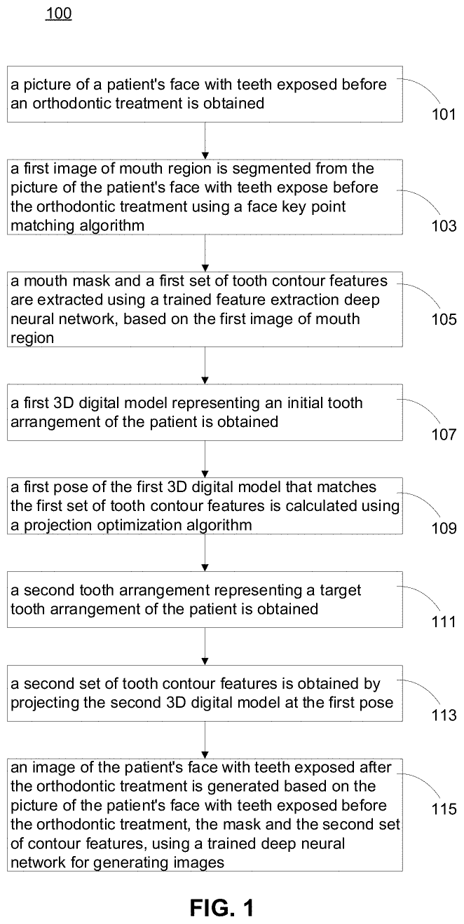

Referring to FIG. 1, it schematically illustrates a method 100 for generating an image of a patient's appearance after an orthodontic treatment using artificial neural network in one embodiment of the present application.

In 101, a picture of a patient's face with teeth exposed before an orthodontic treatment is obtained.

People usually care much about their toothy smiles. Therefore, in one embodiment, the picture of the patient's face with teeth exposed before the orthodontic treatment may be a full face picture of the patient's toothy smile. Such pictures of before and after an orthodontic treatment can clearly show differences before and after the orthodontic treatment. Inspired by the present application, it is understood that the picture of the patient's face with teeth exposed before the orthodontic treatment may be a picture of part of the face, and the angle of the picture may be any other angle in addition to frontal face.

In 103, a first image of mouth region is segmented from the picture of the patient's face with teeth exposed before the dental orthodontic treatment using a face key point matching algorithm.

As compared with a picture of a full face, an image of mouth region has fewer features, as a result, for subsequent processings based on the image of mouth region only, this may simplify computations, may make it easier for artificial neural network(s) to learn, and meanwhile may make the artificial neural network(s) more robust.

For the face key point matching algorithm, reference may be made to the paper “Displaced Dynamic Expression Regression for Real-Time Facial Tracking and Animation” by Chen Chao, Qiming Hou and Kun Zhou in 2014. ACM Transactions on Graphics (TOG) 33, 4 (2014), 43, and the paper “One Millisecond Face Alignment with an Ensemble of Regression Trees” by Vahid Kazemi and Josephine Sullivan in Proceedings of the IEEE Conference on Computer Vision and Pattern Recognition, 1867-1874, 2014.

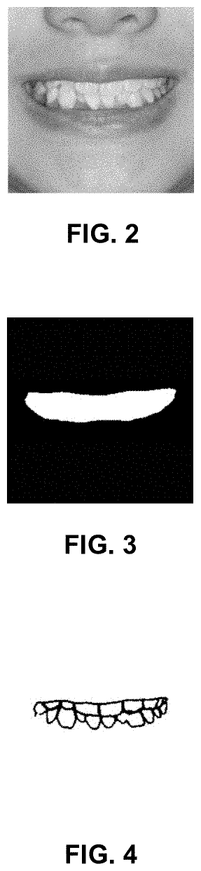

Inspired by the present application, it is understood that the mouth region may be defined in different ways. Referring to FIG. 2, it schematically illustrates an image of mouth region of a patient before an orthodontic treatment in one embodiment of the present application. Although the image of mouth region of FIG. 2 comprises part of the nose and part of the chin, as mentioned above, the mouth region may be reduced or enlarged according to specific needs.

In 105, a mouth mask and a first set of tooth contour features are extracted using a trained feature extraction deep neural network, based on the first image of mouth region.

In one embodiment, the mouth mask may be defined by the inner edge of the lips.

In one embodiment, the mask may be a black and white bitmap, and a part of a picture that is not desired to be displayed can be removed using the mask. Referring to FIG. 3, it schematically illustrates a mouth mask obtained based on the image of mouth region shown in FIG. 2 in one embodiment of the present application.

The tooth contour feature may comprise outlines of each tooth visible in the picture, and it is a two-dimensional feature. In one embodiment, the tooth contour feature may be a tooth contour feature map which only comprises contour information of the teeth. In another embodiment, the tooth contour feature may be a tooth edge feature map which comprises the contour information of the teeth as well as inner side edge features of the teeth, e.g., outlines of spots on the teeth. Referring to FIG. 4, it schematically illustrates a tooth edge feature map obtained based on the image of mouth region shown in FIG. 2 in one embodiment of the present application.

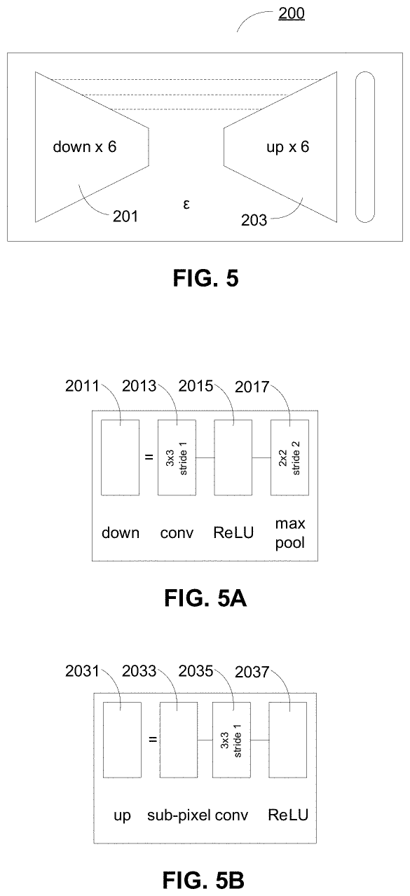

In one embodiment, the feature extraction neural network may be a U-Net network. Referring to FIG. 5, it schematically illustrates the structure of a feature extraction neural network 200 in one embodiment of the present application.

The feature extraction neural network 200 may include six layers of convolution 201 (downsampling) and six layers of deconvolution 203 (upsampling).

Referring to FIG. 5A, each layer of convolution 2011 (down) may include a convolutional layer 2013 (cony), a ReLU activation function 2015 and a maximum pooling layer 2017 (max pool).

Referring to FIG. 5B, each layer of deconvolution 2031 (up) may include a sub-pixel convolutional layer 2033 (sub-pixel), a convolutional layer 2035 (cony) and a ReLU activation function 2037.

In one embodiment, a training set for training the feature extraction neural network may be obtained according to the following: obtaining a plurality of pictures of faces with teeth exposed; segmenting images of mouth region from these pictures of faces; generating corresponding mouth masks and tooth edge feature maps using Photoshop Lasso tool based on the images of mouth region. These images of mouth region and their corresponding mouth masks and tooth edge feature maps may be used as a training set for training the feature extraction neural network.

In one embodiment, to enhance the robustness of the feature extraction neural network, the training set may be augmented by including Gaussian smoothing, rotating, and flipping horizontally etc.

In 107, a first 3D digital model representing the patient's initial tooth arrangement is obtained.

The patient's initial tooth arrangement is a tooth arrangement before the orthodontic treatment.

In some embodiment, the 3D digital model of the patient's initial tooth arrangement may be obtained by directly scanning the patient's jaw. In further embodiments, the 3D digital model representing the patient's initial tooth arrangement may be obtained by scanning a physical model such as a plaster model of the patient's jaw. In yet further embodiment, the 3D digital model representing the patient's initial tooth arrangement may be obtained by scanning an impression of the patient's jaw.

In 109, a first pose of the first 3D digital model that matches the first set of tooth contour features is obtained using a projection optimization algorithm.

In one embodiment, an optimization target of a non-linear projection optimization algorithm may be written as the following Equation (1):

E=Σᵢ^{N}∥\dot{p}ᵢ−pᵢ∥₂ Equation (1)

where \dot{p}ᵢ stands for a sampling point on the first 3D digital model, and pᵢ stands for a point on the outlines of the teeth in the first tooth edge feature map corresponding to the sampling point.

In one embodiment, a correspondence relationship between points on the first 3D digital model and the first set of tooth contour features may be calculated based on the following Equation (2):

$${p}_{i} = {arg min}_{{p}_{j}} {‖ {\dot{p}}_{i} - {p}_{i} ‖}_{2}^{2} \cdot exp ( - < {\dot{t}}_{i} , {t}_{j} {>}^{2} ) \qquad Equation ( 2 )$$

where tᵢ and tⱼ stand for tangential vectors at points pᵢ and pⱼ, respectively.

In 111, a second 3D digital model representing the patient's target tooth arrangement is obtained.

Methods for obtaining a 3D digital model representing a patient's target tooth arrangement based on a 3D digital model representing the patient's initial tooth arrangement is well known in the art and will not be described in detail here.

In 113, the second 3D digital model at the first pose is projected to obtain a second set of tooth contour features.

In one embodiment, the second set of tooth contour features includes outlines of all upper jaw and lower jaw teeth when they are under the target tooth arrangement and at the first pose.

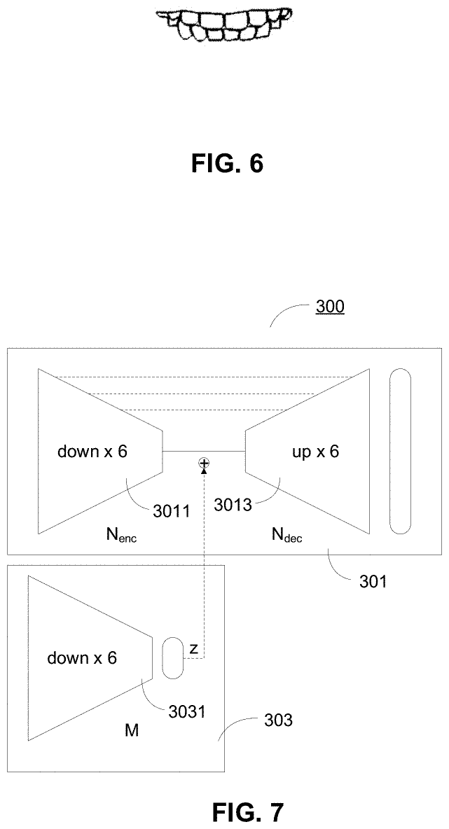

Referring to FIG. 6, it schematically illustrates a second tooth edge feature map in one embodiment of the present application.

In 115, an image of the patient's face with teeth exposed after the orthodontic treatment is generated using a trained deep neural network for generating images, based on the picture of the patient's face with teeth exposed before the orthodontic treatment, the mask and the second set of tooth contour features.

In one embodiment, a CVAE-GAN network may be used as the deep neural network for generating images. Referring to FIG. 7, it schematically illustrates the structure of a deep neural network 300 for generating images in one embodiment of the present application.

The deep neural network 300 for generating images includes a first subnetwork 301 and a second subnetwork 303. A part of the first subnetwork 301 is for processing shapes, and the second subnetwork 303 is for processing textures. Therefore, a part of the picture of the patient face with teeth exposed before the orthodontic treatment or the first image of mouth region, which part corresponds to the mask region, is input to the second subnetwork 303 so that the deep neural network 300 for generating images can generate textures for the part in the image of the patient's face with teeth exposed after the orthodontic treatment. The mask and the second tooth edge feature map are input to the first subnetwork 301 so that the deep neural network 300 for generating images can segment the part of the image of the patient's face with teeth exposed after orthodontic treatment that corresponds to the mask into regions, i.e., teeth, gingival, gaps between teeth, tongue (in the case that tongue is visible) etc.

The first subnetwork 301 includes six layers of convolution 3011 (downsampling) and six layers of deconvolution 3013 (upsampling). The second subnetwork 303 includes six layers of convolution 3031 (downsampling).

A CVAE-GAN network usually includes an encoder, a decoder (can also be called “generator”) and a discriminator (not shown in FIG. 7). In the embodiment that the deep neural network 300 is a CVAE-GAN network, the encoder corresponds to downsampling 3011, which is a common implementation of the encoder. The decoder corresponds to upsampling 3013, upsampling and deconvolution are common implementations of the decoder.

In one embodiment, the deep neural network 300 for generating images may use a differentiable sampling method to facilitate end-to-end training. Reference may be made to “Auto-Encoding Variational Bayes” published by Diederik Kingma and Max Welling in 2013 in ICLR 12 2013 for a similar sampling method.

The training of the deep neural network 300 for generating images may be similar to the training of the abovementioned feature extraction neural network 200, and will not be described in detail any more here.

Inspired by the present application, it is understood that in addition to the CVAE-GAN network, other networks such as cGAN, cVAE, MUNIT or CycleGAN may also be used as the network for generating images.

It is understood that the decoder part 3013 of the first subnetwork 301 can be replaced with any alternative effective decoder (generator), such as a StyleGAN generator. For more details of StyleGAN generator, please refer to “Analyzing and Improving the Image Quality of StyleGAN” CoRR abs/1912.04958 (2019) by Tero Karras, Samuli Laine, Miika Aittala, Janne Hellsten, Jaakko Lehtinen, and Timo Aila.

In one embodiment, the part of the picture of the patient's face with teeth exposed before the orthodontic treatment, which part corresponds to the mask, may be input to the deep neural network 300 for generating images, to generate the part of the image of the patient's face with teeth exposed after the orthodontic treatment, which part corresponds to the mask, and then the image of the patient's face with teeth exposed after the orthodontic treatment is composed based on the picture of the patient's face with teeth exposed before the orthodontic treatment and the part of the image of the patient's face with teeth exposed after the orthodontic treatment, which part corresponds to the mask.

In another embodiment, the mask region of the first image of mouth region may be input to the deep neural network 300 for generating images, to generate the mask region of the image of the patient's face with teeth exposed after the orthodontic treatment, then the second image of mouth region is composed based on the first image of mouth region and the mask region of the image of the patient's face with teeth exposed after the orthodontic treatment, and then the image of the patient's face with teeth exposed after the orthodontic treatment is composed based on the picture of the patient's face with teeth exposed before the orthodontic treatment and the second image of mouth region.

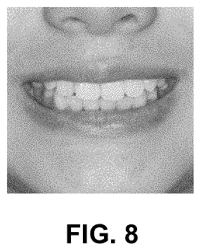

Referring to FIG. 8, it schematically illustrates a second image of mouth region in one embodiment of the present application. Images of patients' faces with teeth exposed after orthodontic treatments generated by the method of the present application are very close to actual outcomes of the orthodontic treatments, and have very high referential value. An image of a patient's face with teeth exposed after an orthodontic treatment is able to help the patient to build confidence on the treatment and meanwhile promote the communications between the orthodontic dentist and the patient.

Inspired by the present application, it is understood that although an image of a patient's full face after an orthodontic treatment can enable the patient to well learn about the treatment effect, this is not requisite. In some cases, a mouth region image of the patient after the dental orthodontic treatment is sufficient to enable the patient to learn about the treatment effect.

While various aspects and embodiments have been disclosed herein, other aspects and embodiments will be apparent to those skilled in the art, inspired by the present application. The various aspects and embodiments disclosed herein are for illustration only and are not intended to be limiting, and the scope and spirit of the present application shall be defined by the following claims.

Likewise, the various diagrams may depict exemplary architectures or other configurations of the disclosed methods and systems, which are helpful for understanding the features and functions that can be included in the disclosed methods and systems. The claimed invention is not restricted to the illustrated exemplary architectures or configurations, and desired features can be achieved using a variety of alternative architectures and configurations. Additionally, with regard to flow diagrams, functional descriptions and method claims, the order in which the blocks are presented herein shall not mandate that various embodiments of the functions shall be implemented in the same order unless otherwise the context specifies.

Unless otherwise specifically specified, terms and phrases used herein are generally intended as “open” terms instead of limiting. In some embodiments, use of phrases such as “one or more”, “at least” and “but not limited to” should not be construed to imply that the parts of the present application that do not use similar phrases intend to be limiting.

We claim:

1. A method for generating image of orthodontic treatment outcome using artificial neural network, comprising:
obtaining a picture of a patient's face with teeth exposed before an orthodontic treatment;
extracting a mouth mask and a first set of tooth contour features from the picture of the patient's face with teeth exposed before the orthodontic treatment using a trained feature extraction deep neural network;
obtaining a first 3D digital model representing an initial tooth arrangement of the patient and a second 3D digital model representing a target tooth arrangement of the patient;
obtaining a first pose of the first 3D digital model based on the first set of tooth contour features and the first 3D digital model;
obtaining a second set of tooth contour features based on the second 3D digital model at the first pose; and
generating an image of the patient's face with teeth exposed after the orthodontic treatment using a trained deep neural network for generating images, based on the picture of the patient's face with teeth exposed before the orthodontic treatment, the mask and the second set of teeth contour features.

2. The method of claim 1, wherein the deep neural network for generating images is a CVAE-GAN network.

3. The method of claim 2, wherein a sampling method used by the CVAE-GAN network is a differentiable sampling method.

4. The method of claim 1, wherein the deep neural network for generating images includes a decoder, where the decoder is a StyleGAN generator.

5. The method of claim 1, wherein the feature extraction deep neural network is a U-Net network.

6. The method of claim 1, wherein the first pose is obtained using a nonlinear projection optimization method based on the first set of tooth contour features and the first 3D digital model, and the second set of tooth contour features are obtained by projecting the second 3D digital model at the first pose.

7. The method of claim 1, further comprising: segmenting a first image of mouth region from the picture of the patient's face with teeth exposed before the orthodontic treatment using a face key point matching algorithm, where the mouth mask and the first set of tooth contour features are extracted from the first image of mouth region.

8. The method of claim 7, wherein the picture of the patient's face with teeth exposed before the orthodontic treatment is a picture of the patient's full face.

9. The method of claim 7, wherein the contour of the mask matches the contour of the inner side of the lips in the picture of the patient's face with teeth exposed before the orthodontic treatment.

10. The method of claim 9, wherein the first set of tooth contour features comprise outlines of teeth visible from the picture of the patient's face with teeth exposed before the orthodontic treatment, and the second set of tooth contour features comprise outlines of the second 3D digital model at the first pose.

11. The method of claim 10, wherein the tooth contour features are a tooth edge feature map.

12. The method of claim 2, further comprising: segmenting a first image of mouth region from the picture of the patient's face with teeth exposed before the orthodontic treatment using a face key point matching algorithm, where the mouth mask and the first set of tooth contour features are extracted from the first image of mouth region.

13. The method of claim 3, further comprising: segmenting a first image of mouth region from the picture of the patient's face with teeth exposed before the orthodontic treatment using a face key point matching algorithm, where the mouth mask and the first set of tooth contour features are extracted from the first image of mouth region.

14. The method of claim 4, further comprising: segmenting a first image of mouth region from the picture of the patient's face with teeth exposed before the orthodontic treatment using a face key point matching algorithm, where the mouth mask and the first set of tooth contour features are extracted from the first image of mouth region.

15. The method of claim 5, further comprising: segmenting a first image of mouth region from the picture of the patient's face with teeth exposed before the orthodontic treatment using a face key point matching algorithm, where the mouth mask and the first set of tooth contour features are extracted from the first image of mouth region.

16. The method of claim 6, further comprising: segmenting a first image of mouth region from the picture of the patient's face with teeth exposed before the orthodontic treatment using a face key point matching algorithm, where the mouth mask and the first set of tooth contour features are extracted from the first image of mouth region.
